(19) United States
(12) Patent Application Publication
(10) Pub. No.: US 2024/0316281 A1
(43) Pub. Date: Sep. 26, 2024

(54) A PREFILLED SYRINGE CONTAINING A STERILE ULTRACONCENTRATED HYDROGEL

(71) Applicant: Vitrean Inc., Palo Alto, CA (US)

(72) Inventors: Ira H. Schachar, Palo Alto, CA (US); Ronald A. Schachar, Palo Alto, CA (US)

(73) Assignee: Vitrean Inc., Palo Alto, CA (US)

(21) Appl. No.: 18/554,197

(22) PCT Filed: Mar. 31, 2022
(86) PCT No.: PCT/US2022/071458

§ 371 Date: Oct. 5, 2023

Related U.S. Application Data
(60) Provisional application No. 63/171,232, filed on Apr. 6, 2021.

(51) Int. Cl. A61M 5/315; A61K 9/06; A61K 47/36; A61M 5/32

(52) U.S. Cl. CPC A61M 5/31511; A61K 9/06; A61K 47/36; A61M 5/3286

(57) ABSTRACT

A prefilled syringe containing a sterile ophthalmic pharmaceutical composition comprising an ultraconcentrated hydrogel and a pharmaceutically acceptable carrier, the prefilled syringe capable of storing the ultraconcentrated hydrogel and injecting the ultraconcentrated hydrogel with a reduced injection force into a biologic tissue.

### BACKGROUND OF THE INVENTION

### Field of the Invention

The present invention relates in general to a pre-filled syringe containing a sterile ophthalmic pharmaceutical composition comprising an ultraconcentrated hydrogel and a pharmaceutically acceptable carrier.

### Discussion of the Background

Hydrogels are three dimensional networks of hydrophilic polymer chains that, due to their hydrophilic nature, are able to swell and retain a significant fraction of water within their structure. Over the past 50 years, they have been extensively used in industrial and medical applications, with particularly high usage in ophthalmology, plastic surgery, dermatology, and orthopedics. There are a wide range of hydrophilic polymers that have been used to synthesize hydrogels including natural, semi-synthetic, and synthetic polymers. As the concentration of these hydrophilic polymers in the hydrogel increase, the physiochemical properties of the hydrogel change. Standard hydrogels (storage modulus G′<150 Pa) can be injected through a standard syringe and needle. However, for a number of indications, smaller microneedles are desired. Therefore, specially designed syringes capable of withstanding high structural forces have been developed (US20200246555A1). The focus of such syringes has been to withstand high injection forces to prevent syringe breakage or needle pop off (NPO).

Ultraconcentrated hydrogels have many potential benefits over standard hydrogels in terms of resident time, drug delivery, duration of action, and therapeutic effect. Such ultraconcentrated hydrogels have an extremely high storage moduli G′, often greater than 500 Pa, far exceeding that of standard hydrogels. Counterintuitively, despite this high storage modulus, ultraconcentrated hydrogels can be passed through a needle when sufficient force is applied. In these instances, the ultraconcentrated hydrogel undergoes shear thinning thereby allowing passage through a needle. Unfortunately, high forces applied by the user are undesirable when precise delivery to biologic tissues is desired such as occurs during intravitreal injections into the eye or injections into the joint space. This inability to deliver ultraconcentrated hydrogels to the biologic tissue of interest has prevented the development of ultraconcentrated hydrogels in general, and, more specifically prevented development of ultraconcentrated hydrogels with ophthalmic and/or orthopedic formulations. It is therefore critically necessary to develop pharmaceutical compositions comprising an ultraconcentrated hydrogel and design syringes that are capable of storing such ultraconcentrated hydrogels and delivering such ultraconcentrated hydrogels to a biologic tissue with reduced injection force required by the user.

Syringe coatings have been one approach to reducing syringe injection force. For ultraconcentrated hydrogels, such syringe coatings are insufficient to sufficiently reduce injection forces and allow for precision delivery. Standard syringe coatings pose an additional issue for syringes containing ultraconcentrated hydrogels. Ultraconcentrated hydrogels are highly negatively charged. This strong negative charge causes ionic attraction between the syringe coating and the ultraconcentrated hydrogel. This attractive force results in leaching of the syringe coating into the ultraconcentrated hydrogel creating subvisible and visible particulates. Such particulates are poorly tolerated when injected into biologic tissue. An interrelated issue is that of storage. Leaching is dependent on the duration of contact between the ultraconcentrated hydrogel. Ultraconcentrated hydrogels cannot be drawn into a syringe by the user due to their high storage modulus. Instead, syringes must be prefilled using special techniques, and the ultraconcentrated hydrogel stored within the syringe until use. Because of potentially long storage times, particulates from the syringe coating may rapidly develop within the syringe.

What is needed in the art is a syringe capable of minimizing the injection force required for delivery of ultraconcentrated hydrogels through a needle to precise biologic locations.

Moser et al. US2020/0246555A1 discloses a syringe body for a syringe for injecting a highly viscous medium which has a hollow cylindrical configuration, forms a chamber configured to receive the highly viscous medium, and includes: a distal end portion; a proximal end portion having an opening through which a piston rod arrangement is insertable into the chamber; and a Luer lock connector formed at the distal end portion, the Luer lock connector having an outer cone with a further opening configured to dispense the highly viscous medium and a sleeve-shaped portion with an inner thread.

WO2019124938 discloses a method for manufacturing a pre-filling type syringe comprising a hyaluronic acid hydrogel containing a local anesthetic.

WO2012118194A1 discloses a prefilled syringe that can stably store a highly-pure sodium hyaluranate aqueous solution.

Sakhrani et al. U.S. Pat. No. 7,431,989 discloses a method for preparing one or more lubricated surfaces of an article to reduce the break-out force and sliding frictional force.

Sakhrani et al. U.S. Pat. No. 7,553,529 discloses a method for preparing one or more lubricated surfaces of an article to reduce the break-out force and sliding frictional force.

Sakhrani et al. U.S. Pat. No. 8,084,103 discloses a method for increasing the hydrophobic characteristics of a surface.

Thornton et al. U.S. Pat. No. 9,133,412 discloses a method for preparing one or more lubricated surfaces of an article to reduce the break-out force and sliding frictional force. A lubricant is applied to one or more surfaces, and the lubricant-coated surface is treated by exposing the surface to an energy source, wherein the energy source is an ionizing gas plasma at about atmospheric pressure, gamma radiation, or electron beam radiation.

Sakhrani et al. U.S. Pat. No. 7,431,989 a method for preparing one or more lubricated surfaces of an article to reduce the break-out force and sliding frictional force. A lubricant is applied to one or more surfaces, and the lubricant-coated surface is treated by exposing the surface to an energy source, wherein the energy source is an ionizing gas plasma at about atmospheric pressure, gamma radiation, or electron beam radiation.

Felts et al. U.S. Pat. No. 9,878,101 A package including a vessel made of cyclic olefin polymer (COP) resin, a CVD coating on a vessel, and a medicament comprising at least one protein, peptide, and/or DNA sequence is disclosed.

Braca et al. U.S. Pat. No. 5,536,572 discloses an olefin polymer composition which comprises: (a) from 0.3 to 3% of a complex of general formula: wherein R is an amine; Me is bismuth or antimony; X is chlorine or bromine; y is a number from 0.3 to 4.0; (b) from 0.1 to 1% of poly(1,4-diisopropylbenzene); (c) from 0.1 to 0.5% of an organic epoxide; the balance to 100% being an olefin polymer.

Haselkorn WO9917821A1 discloses a loaded single use disposable device for introducing a viscoelastic substance into the intraocular space of a patient.

Schachar et al. U.S. Pat. No. 10,849,855 describes methods and devices for treating a retinal detachment by injecting a substance into a vitreous cavity of the eye.

### SUMMARY OF THE INVENTION

This disclosure relates to a prefilled syringe containing a sterile ophthalmic pharmaceutical composition comprising an ultraconcentrated hydrogel in a pharmaceutically acceptable carrier. More specifically it relates to a prefilled cyclic-olefin polymer syringe uniquely designed to reduce injection force of ultraconcentrated hyaluronic acid into a biologic tissue.

Four primary features affect force requirements when injecting ultraconcentrated hydrogels. These are (1) the concentration and composition of the hydrogel, (2) the dimensions of the syringe, barrel and plunger, (3) the internal coating on the syringe, and (4) the internal diameter of the needle. The present invention is in regard to an ophthalmic pharmaceutical composition comprising an ultraconcentrated hydrogel and a pharmaceutically acceptable carrier contained within a prefilled syringe that are capable of both storing the ultraconcentrated hydrogels and injecting the ultraconcentrated hydrogels with reduced injection force.

In some exemplary embodiments provided according to the present invention, a syringe capable of storing an ultraconcentrated hydrogel and capable of injecting said hydrogel through a needle with an injection force that is below a clinical safety threshold The syringe is a prefilled syringe that is capable of storing the ultraconcentrated hydrogel. The prefilled syringe limits water vapor permeability into the ultraconcentrated hydrogel. The internal portions of the syringe barrel have a covalently attached coating to reduce friction between the syringe plunger and the syringe barrel. The covalently attached coating exhibits low leachables into the ultraconcentrated hydrogel. The syringe barrel has finger flanges to provide grip to the user. The cross-sectional area of the plunger tip (and/or barrel diameter) is less than 8 mm, preferably less than 6 mm, more preferably less than 4 mm, most preferably less than 3.5 mm. The cross-sectional area of the thumb press is 8-fold greater than, preferably 10-fold greater than, more preferably 15-fold greater than, more preferably 20-fold greater than, most preferably 25-fold greater than the cross-sectional area of the plunger tip (and/or barrel diameter). The syringe contains a method of attaching a needle to the syringe, such as a Leur lock.

### BRIEF DESCRIPTION OF THE DRAWINGS

FIG. 1. Injection force testing of different syringes, coatings, and needles;

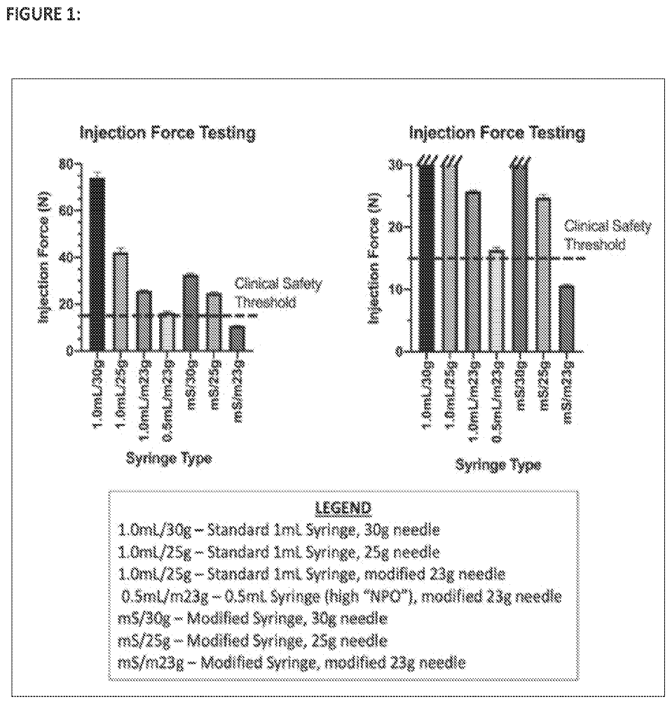

FIG. 2. Effect of cross-linked syringe barrel coating on particulates;

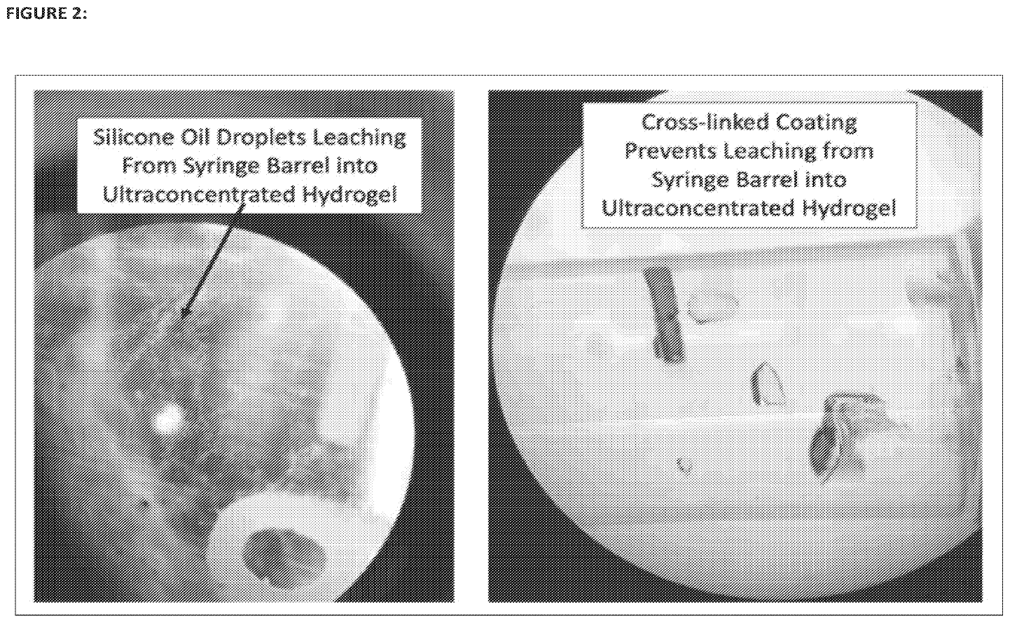

FIG. 3. Exemplary syringe designed to reduce injection force;

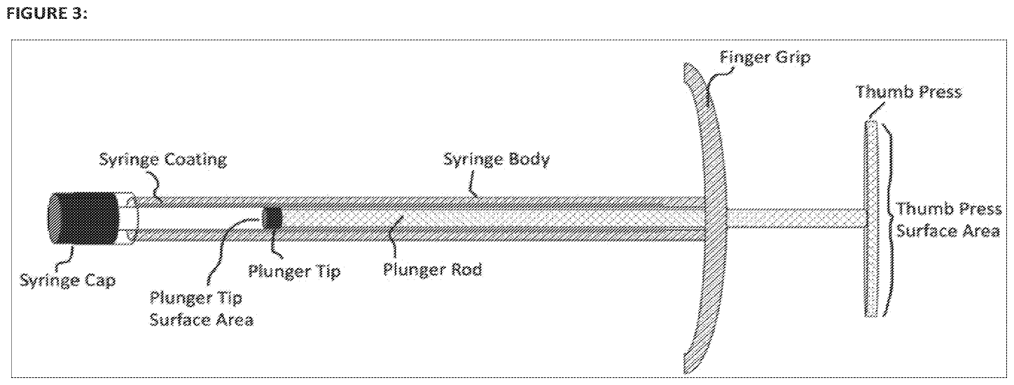

FIG. 4. Exemplary syringe designed to reduce injection force with a leur lock;

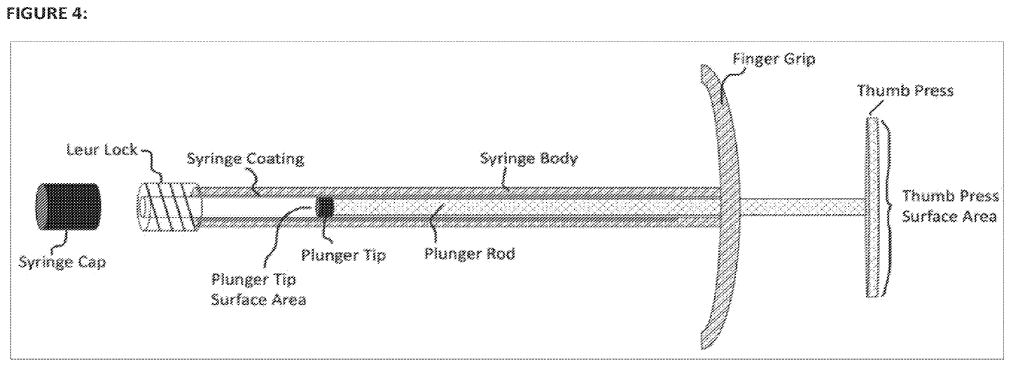

FIG. 5. Exemplary syringe designed to reduce injection force with a leur lock and an ultrathin-walled needle;

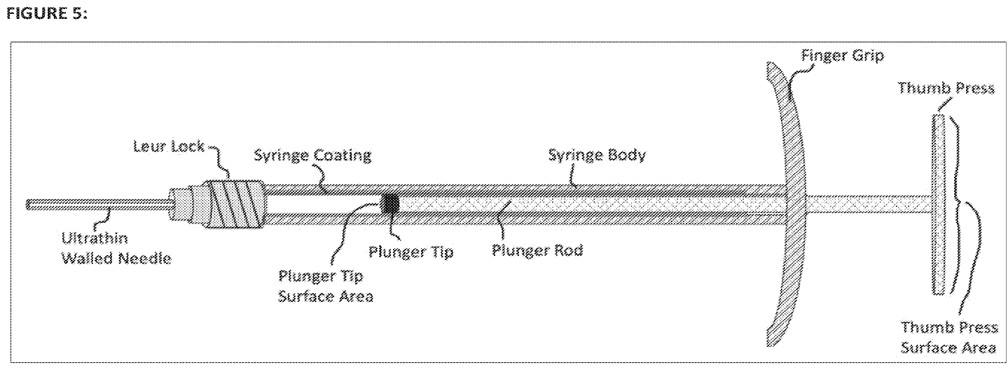

FIG. 6. Ultra-thin walled needle;

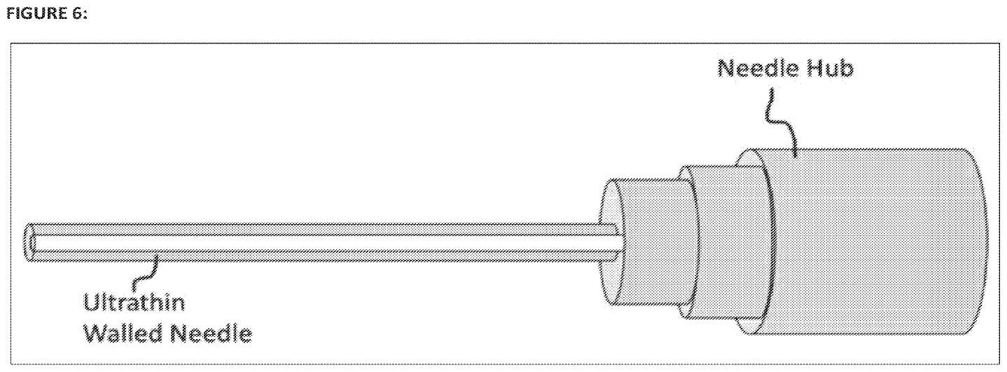

FIG. 7. Exemplary syringe designed to reduce injection force with a leur lock, an ultrathin-walled needle, and a needle lock; and

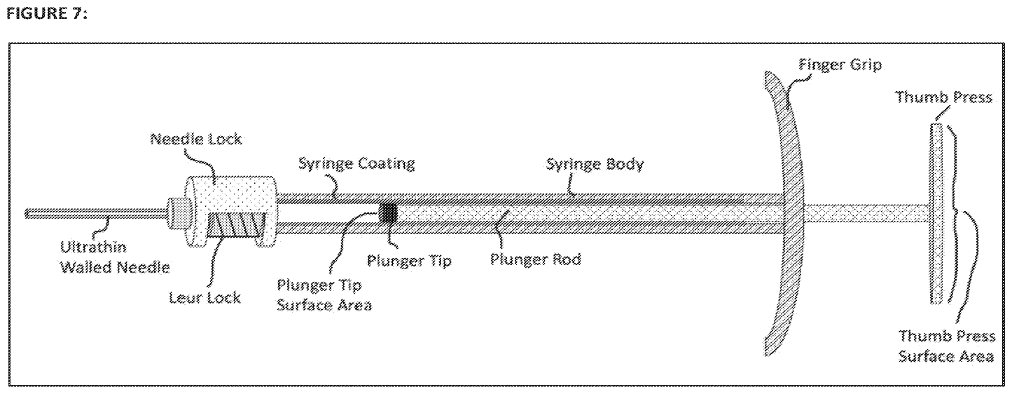

FIG. 8. Example of needle lock added to a syringe and ultrathin walled needle.

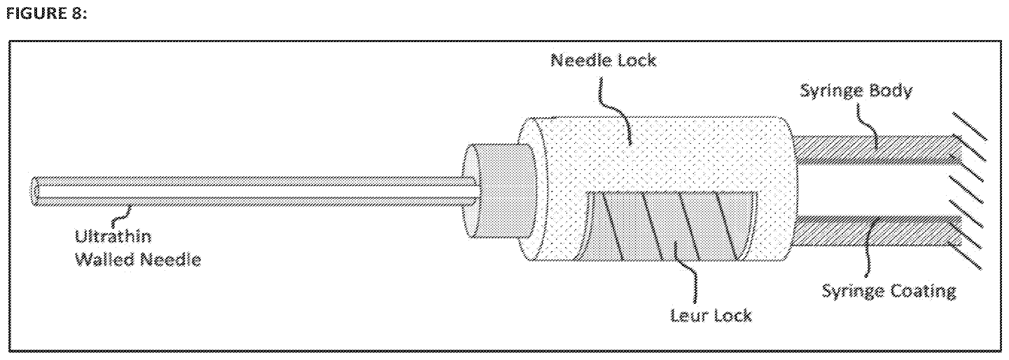

### DETAILED DESCRIPTION OF THE PREFERRED EMBODIMENTS

Hydrogel as used herein refers to a network of polymer chains that are hydrophilic and are dispersed within an aqueous medium. A three-dimensional structure can result from the hydrophilic polymer chains being held together by cross-links, which may be covalent or ionic crosslinks, such as hydrogen bonds between the hydrophilic polymer side chains or hydrogen bonds formed with water molecules of the aqueous medium. These cross-links may be permanently formed or may be transitory. Hydrogels may be described based on their polymer backbone (e.g. hyaluronic acid, carboxymethylcellulose), concentration, and molecular weight. Hydrogels referred herein may be contain one or more polymer backbone, concentration, and/or molecular weight.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Although various illustrative embodiments are described, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

In some variations, the hydrophilic polymer contained within the syringe may be a synthetic, semi-synthetic or natural polymer. Suitable hydrophilic polymers may include any of natural gums, starches, pectins, agar-agar, gelatin, mechanical and thixotropic agents, polyurethanes, acrylic polymers, latex, styrene/butadiene, polyvinyl alcohol (PVA), cellulosics (cellulose acetate), cellulose triacetate, cellulose propionate, cellulose acetate propionate (CAP), cellulose acetate butyrate (CAB), nitrocellulose, cellulose sulfate, methyl cellulose, ethylcellulose, ethyl methyl cellulose, hydroxypropyl cellulose (HPC), hydroxyethyl methyl cellulose, ethyl hydroxyethyl cellulose, carboxymethyl cellulose (CMC), hydroxyl methylcellulose (HMC), hydroxyethyl cellulose, hydroxypropyl methylcellulose (HPMC), chemically modified cellulose macromolecules), sulfonates, gums (guar, xanthan, cellulose, locust bean, acacia), saccharides (carrageenan, pullulan, konjac, alginate), proteins (casein, collagen, albumin), modified castor oil, organosilicones (silicone resins, dimethicones, modified silicones), synthetic hydrogels (polyvinyl alcohol, sodium polyacrylate, acrylate polymers, and copolymers), organogels, xerogels, natural hydrogels (agarose, methylcellulose, glycosaminoglycan such as hyaluronic acid (also known as sodium hyaluronate or hyaluronan), chondroitin sulfate, heparosan), modified hydrogels (discussed in detail below), cross-linked hydrogels, polyionic polymers, and nanocomposite hydrogels. In some variations, the hydrophilic polymer may be a glycosaminoglycan.

In some variations, the hydrophilic polymer may be a combination of one or more of the aforementioned hydrophilic polymers. In other variations, the hydrophilic polymer may contain polymers of different molecular weights. In some variations there may be a low molecular weight hydrophilic polymer that is combined with a high molecular weight hydrophilic polymer. In some variations these different molecular weight hydrophilic polymers are the same polymer, while in other variations they are different polymers.

In some variations, the hydrophilic polymer may be a hyaluronic acid (also known as sodium hyaluronate or hyaluronan). In some variations, the hydrophilic polymer may be a modified form of hyaluronic acid. In some instances, the hydrophilic polymer is a collection of hyaluronic acid at different molecular weights. In some instances, the hydrophilic polymer contains hyaluronic acid and heparaosan. In some instances, the hydrophilic polymer is hyaluronic acid with a molecular weight greater than 700 kDa. In some instances, the hydrophilic polymer is hyaluronic acid with a molecular weight greater than 1.5 MDa. In some instances, the hydrophilic polymer is hyaluronic acid with a molecular weight greater than 4 MDa. In some instances, the hydrophilic polymer is hyaluronic acid with a molecular weight greater than 6 MDa. In some instances, the hydrophilic polymer is hyaluronic acid with mixed molecular weights both above 4 MDa and below 2 MDa. the hydrophilic polymer is hyaluronic acid with mixed molecular weights both above 4 MDa and below 800 kDa. the hydrophilic polymer is hyaluronic acid with mixed molecular weights both above 4 MDa and below 100 kDa. In some instances, the hyaluronic acid has been extracted from rooster combs. In some instances, the hyaluronic acid has been engineered using bacteria. In some instances, the hydrophilic polymer is hyaluronic acid derived from rooster combs that has a molecular weight of at or above 2.5 MDa, at or above 3.0 MDa, at or above 3.5 MDa, at or above 4.0 MDa, at or above 4.5 MDa, at or above 5.0 MDa, at or above 5.5 MDa, at or above 6.0 MDa, at or above 6.5 MDa, at or above 7.0 MDa, at or above 7.5 MDa. In some instances, the hyaluronic acid has a molecular weight of 6.7 MDa. In some instances, the molecular weight is at or below 6.0 MDa, at or below 8 MDa, at or below 10 MDa. In some instances, the hyaluronic acid has a narrow distribution around a specific molecular weight. In some instances, this range is less than 20%, is less than 10%, is less than 5%, is less than 1%, is less than 0.5% of the average molecular weight. In some instances, the hyaluronic acid has an intrinsic viscosity at or above 1.0 m³/kg, at or above 2.0, m³/kg at or above 3.0 m³/kg, at or above 4.0 m³/kg, at or above 5.0 m³/kg, at or above 6.0 m³/kg. In some instances, the hyaluronic acid has an intrinsic viscosity at or below 10.0 m³/kg, at or below 8.0 m³/kg, at or below 6.0 m³/kg, at or below 4.0 m³/kg. In some instances, the hyaluronic acid has an intrinsic viscosity between 4.0 and 5.0 m³/kg. In some instances, the hyaluronic acid has an intrinsic viscosity between 5.0 m³/kg and 5.5 m³/kg. In some instances, the hyaluronic acid has an intrinsic viscosity between 5.5 m³/kg and 6.0 m³/kg In some instances, the hyaluronic acid bas an intrinsic viscosity of 4.2. In some instances, the hyaluronic acid has an intrinsic viscosity of 4.7. In some instances, the hyaluronic acid has an intrinsic viscosity of 5.0 m³/kg. In some instances, the hyaluronic acid is crosslinked.

For any of the aforementioned substances, covalent or non-covalent modifications can be made. Common covalent modifications that can be added to any of the aforementioned substances include, but are not limited to, maleimide addition, methacrylate addition, aldehyde addition, thiol addition, furan addition, amine addition, carboxyl addition, epoxide addition, PEGylation, hydrazide addition, NHS ester addition, siloxane addition, and tyramine addition. In some embodiments, the hydrogel is cross-linked. In some embodiments, the hydrophilic polymer is hyaluronic acid. In these instances, the hyaluronic acid may be modified with the aforementioned modifications.

In some variations, the hydrogel is ultraconcentrated at or above 5% w/w, at or above 7.5%, at or above 10%, at or above 12.5%, at or above 15%, at or above 17.5%, at or above 20%, at or above 22.5%, at or above 25%, at or above 27.5%, at or above 30%, at or above 32.5%, at or above 35%, at or above 37.5%, at or above 40%, at or above 45%, at or above 50%, at or above 55%, at or above 60%. In some variations, the hydrogel is hyaluronic acid and is ultraconcentrated to at or above 5% w/w, at or above 7.5% w/w, at or above 10%, at or above 12.5%, at or above 15%, at or above 17.5%, at or above 20%, at or above 22.5%, at or above 25%, at or above 27.5%, at or above 30%, at or above 32.5%, at or above 35%, at or above 37.5%, at or above 40%, at or above 45%, at or above 50%, at or above 55%, at or above 60%.

The hydrogel may contain any number of aqueous excipients. Such aqueous excipient may be acidic, neutral, or basic. In some instances, the pH may be physiologic. In some instances, the pH may be less than 5. In some instances, the pH may be greater than 8. In some instances, the pH may be between 5 and 8. In some instances, the pH may be between 6 and 8. In some instances, the pH may be between 7 and 7.4.

In some variations, the aqueous excipient is a physiologic saline solution. In some instances, the aqueous excipient may be buffered. In some instances, the buffer a simple buffer. Examples include, but are not limited to, citric acid, phosphate, acetic acid, CHES (N-cyclohexyl-2-aminoethanesulfonic acid), borate, or boronic acid. In other instances, the buffer is TAPS ([tris(hydroxymethyl)methylamino]propanesulfonic acid), Bicine (2-(bis(2-hydroxyethyl)amino)acetic Tris (tris(hydroxymethyl)aminomethane) or 2-(hydroxymethyl)propane-1,3-diol), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]ethanesulfonic acid), MOPS (3-(N-morpholino)propanesulfonic acid), PIPES (piperazine-N,N′-bis(2-ethanesulfonic acid)), Cacodylate (dimethylarsenic acid), MES (2-N-morpholino)ethanesulfonic acid). In some instances, one or more of the aforementioned buffers are combined. In some instances, the aqueous excipient contains additional salts. In some instances, these salts include sodium chloride, potassium chloride, calcium chloride, magnesium chloride, sodium acetate, disodium hydrogen phosphate dihydrate, sodium dihydrogen phosphate dihydrate, or sodium citrate.

In some instances, the aqueous excipient contains a carbohydrate. In some instances, the carbohydrate is a monosaccharide. In some instances, the carbohydrate is a disaccharide. In some instances, the carbohydrate is a polysaccharide. In some instances, the carbohydrate is glucose. In some instances, the glucose is present at a physiologic concentration. In some instances, glucose is present at a supraphysiologic concentration. In some instances, glucose is present at a concentration at about 1 uM, at about 10 uM, at about 100 uM, at about 1 mM, at about 10 mM, at about 100 mM, or above about 1M.

In some instances, the osmolarity of the aqueous excipient is physiologic. In some instances, the osmolarity is between 250 and 400 mosm/kg. In some instances, the osmolarity is between 300 and 360 mosm/kg.

In some instances, the ultraconcentrated hydrogel contains one or more substances to reduce hyaluronic acid breakdown. In some instances, the ultraconcentrated hydrogel contains one or more substances to reduce degradation. In some instances, the ultraconcentrated hydrogel contains one or more substances to enhance storage.

In some variations, the syringe contains one or more biologically active substance in addition to the ultraconcentrated hydrogel.

In some variations, one or more biologically active substances is one or more antibodies, one or more proteins, one or more peptides, one or more aptamers, one or more small molecules, one or more enzymes, one or more living cells, and/or one or more cell-based products or materials. In some instances, these biologically active substances (such as antibodies, proteins, peptides, aptamers, small molecules, cells or cell-based products) may be added to enhance the biologic function of the hydrogel.

In some variations, one or more biologically active substances may have biological activity inhibiting cell death and/or protecting nerve activity. In some variations, one or more biologically active substances may have biological activity to limit photoreceptor cell death. In some variations, one or more biologically active substances may have biological activity to limit vessel growth. In some variations, one or more biologically active substances may have biological activity to limit vascular leakage. In some variations, one or more biologically active substance may have biological activity to limit inflammation. In some variations, one or more biologically active substances may have biologic activity to reduce complement activation. In some variations, one or more biologically active substances may have biological activity to reduce scarring. In some variations, one or more biologically active substances may have biological activity to reduce proliferative vitreoretinopathy. In some variations, one or more biologically active substances may have biological activity to reduce intraocular pressure. In some variations, one or more biologically active substances may increase the resident time of the hydrogel. In some variations, one or more biologically active substances may reduce the resident time of the hydrogel.

In some instances, one or more biologically active substances targets one or more molecular targets. In some instances, the one or more molecular targets include vascular endothelial growth factor (VEGF), tumor necrosis factor (TNF), glycoprotein IIb/IIIa (GPIIb/IIIa), B-cell activating factor (BAFF), interluekin-2 receptor alpha chain (CD25), CAMPATH-1 antigen (CD52), proprotein convertase subtilisin/kexin type 9 (PCSK9), programmed death-ligand 1 (PD-L1), Receptor tyrosine-protein kinase erbB-2 (CD350, HER2/neu), clostridium difficile toxin B, B-lymphocyte antigen CD19 (CD19), TNFRSF8 (CD30), interleukin 17 receptor A (IL17RA), interleukin 1 beta (IL-1B), Glutamate carboxypeptidase II (GCPII), epidermal growth factor receptor (EGFR), interleukin-2 receptor (IL-2R), cyclic ADP ribose hydrolase (CD38), receptor activator of nuclear factor kappa-B ligand (RANKL), GD2, interleukin 4 receptor (IL4R), complement component 5 (C5 gene), complement factor D (CFD), SLAM family member 7 (SLAMF7), B-lymphocyte antigen (CD20), interleukin-17A (IL17A), interleukin 5 (ILS), programmed cell death protein 1 (PD-1), platelet-derived growth factor receptor A (PDGFRA), immunoglobulin E (IgE), interleukin 6 (IL-6), interleukin 6 receptor (IL6R), interleukin 12 (IL-12), interleukin-23 (IL-23), CD22, CD33, CD4, interleukin-5 receptor alpha subunit, fibroblast growth factor 23 (FGF23), calcitonin gene-related peptide (CGRP), complement cascade (Clq, Clr, Cls, MBL, MASP, C4b2a, Factor B, Factor D, C3, C3bBb, C3a, C5a, CS convertase, C5), anti-high temperature requirement Al (aHtrA1), membrane attack complex (MAC), angiotension-1 (Ang-1) and/or angiotension-2 (Ang-2). In some instances, the one or more biologically active substances simultaneously targets one or more of the aforementioned molecular targets.

In some instances, one or more of the biologically active substances is an antibody-based therapeutic. In some instances, the antibody-based therapeutic one or more monoclonal antibody, Fab fragment, scFv fragment, diabody, minibody, triabody, tetrabody, tandem di-scFV, tandem tri-scFv, bispecifc diabody, (scFV)₂, sc(Fv)₂, F(ab)₂ fragment, a trifunctional antibody, a chemically linked F(ab)₂, and/or a bi-specific T cell engager (BiTE). In some instances, the antibody-based therapeutic can simultaneously bind two or more different targets. In some instances, the therapeutic is a bispecific monoclonal antibody. In some instances, the therapeutic is a bispecific F(ab)2 fragment. In some instances, the biologically active substance is an Fc-therapeutic. In some instances, the Fc-therapeutic therapeutic can simultaneously bind two or more different targets. In some instances, the bispecific antibody-based therapeutic targets vascular endothelial growth factor (VEGF) and angiopoietin-2 (Ang-2). In some instances, the Fc-based therapeutic targets vascular endothelial growth factor (VEGF) and angiopoietin-2 (Ang-2). In some instances, one or more of the biologically active substances is one or more biosimilars of an antibody-based therapeutic.

In some instances, one or more of the biologically active substances in the ultraconcentrated hydrogel is 3F8, 8H9, abicipar, avacincaptad pegol, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, amivantamab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atidortoxumab, atinumab, atoltivimab, atoltivmab/maftivimab, atorolimumab, avelumab, azintuxizumab vedotin, bapineuzumab, basiliximab, bavituximab, BCD-100, bectumomab, begelomab, belantamab mafodotin, belimumab, bermarituzumab, benralizumab, berlimatoxumab, bermekimab, bersanlimab, bertilmumab, besilesomab, bevacizumab, bezlotuxumab, biciromab, bimagrumab, bimekizumab, birtamimab, bivatuzumab, bleselumab, blinatumomab, blotuvetmab, blosozumab, bocoizumab, brazikumab, brentuximab, briakinumab, brodlalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox. Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab], Dinutuximab, Dinutuximab beta, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegaptanib, Pegcetacoploan, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, REGN-EB3, Relatlimab, Remtolumab, Reslizumab, Rilotumumab, Rinucumab, Risankizumab, Risuteganib, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab Tezepelumab, TGN1412, Tibulizumab, Tildrakizumab . Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBS07, Tregalizumab, Tremelimumab, Trevogrumab, Tucotuzumab celmoleukin, Tuvirumab Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab 1 Vesencumab, Visilizumab, Vobarilizumab, Volociximab, Vonlerolizumab, Vopratelimab, Vorsetuzumab mafodotin, Votumumab, Vunakizumab, and/or Xentuzumab.

In some instances, the concentration of one or more of the biologically active substances in the ultraconcentrated hydrogel is at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, the biologically active substance is bevacizumab. In some instances, the ultraconcentrated bydrogel contains bevacizumab at a concentration of 25 mg/mL. In some instances, the ultraconcentrated hydrogel contains bevacizumab at a concentration at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the pharmaceutical product is ranibizumab. In some instances, the final hydrogel contains ranibizumab at a concentration of 6 mg/mL. In some instances, the ultraconcentrated hydrogel contains ranibizumab at a concentration at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the final hydrogel contains ranibizumab at a concentration of 10 mg/mL. In some instances, the biologically active substance is aflibercept. In some instances, the ultraconcentrated hydrogel contains aflibercept at a concentration at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the final hydrogel contains aflibercept at a concentration of 40 mg/mL. In some instances, the biologically active substance is brolucizumab. In some instances, the ultraconcentrated hydrogel contains brolucizumab at a concentration at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/ml, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/ml. In some instances, final hydrogel contains brolucizumab at a concentration of 120 mg/mL. In some instances, the final hydrogel contains pegaptanib at a concentration of 3.47 mg/mL. In some instances, the final hydrogel contains pegaptanib at a concentration at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more of the biologically active substances is a small molecule. In some instances, the small molecule decreases vascular endothelial growth factor (VEGF) production. In some instances, the small molecule is a receptor tyrosine kinase inhibitor. In some instances, the pharmaceutical product is carbozantinib, pazopanib, sunitinib, axitinib, levatinib, sorafenib, risuteganib, or regorafenib. In some instances, the concentration of the small molecule in the ultraconcentrated hydrogel is at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more biologically active substances is an inhibitors of apoptosis, such as, but not limited to, hydrophilic bile acids (UDCA or TUDCA), anti-FAS-ligand antibodies, MET12 or a fragment thereof, Faim2 or a fragment thereof, caspase inhibitors, or neuroprotective agents such as, but not limited to, MCP-1 antagonist, TNF-alpha antagonist, IL-1 beta antagonist, or a bFGF mimetic, may be ingredients added to reduced cell death in the environment where the highly concentrated hydrogel composition may be applied. Some possible biologically active molecules suitable for use in these compositions are described, e.g., in U.S. Pat. Nos. 7,811,832; 9,192,650; 8,343,931; 9,549,895 and 9,724,357.

In some instances, one or more biologically active substances reduces scar formation. In some instances, one or more biologically active substances reduces proliferative vitreoretinopathy. In some instances, one or more biologically active substances is retinoic acid. In some instances, the retinoic acid is initially dissolved in ethanol. In some instances, the retinoic acid is dissolved in DMSO. In some instances, the concentration of retinoic acid in the final hydrogel is at or above 1 ug/mL, at or above 5 ug/mL, at or above 10 ug/mL, at or above 50 ug/mL, at or above 100 ug/mL, at or above 200 ug/mL, at or above 500 ug/mL, at or above 1 mg/mL, at or above 5 mg/mL, at or above 10 mg/mL, at or above 50 mg/mL, at or above 100 mg/mL. In some instances, the biologically active substance is methotrexate. In some instances, the concentration of methotrexate in the final hydrogel is at or above 1 ug/mL, at or above 5 ug/mL, at or above 10 ug/mL, at or above 50 ug/mL, at or above 100 ug/mL, at or above 200 ug/mL, at or above 500 ug/mL, at or above 1 mg/mL, at or above 2 mg/mL, at or above 5 mg/mL, or at or above 10 mg/mL. In some instances, one or more biologically active substances is fluorouracil (5-FU). In some instances, the 5-FU concentration in the final hydrogel is around 200 ug/mL. In some instances, one or more biologically active substances is a corticosteroid. In some instances, the steroid is a hydrocortisone type (e.g. hydrocortisone, hydrocortisone acetate, cortisone acetate, tixocortol pivalate, prednisolone, methylprednisolone, prednisone). In some instances, the steroid is an acetonide (e.g. amicinonide, budesonide, desonide, fluocinolone acetonide, fluocinonide, halcinonide, triamcinolone acetate). In some instances, the steroid is a betamethasone type (e.g. beclomethasone, betamethasone, dexamethasone, fluocortolone, halometasone, mometasone). In some instances, the steroid is an ester. In some instances, the steroid is halogenated (e.g. alclometasone dipropionate, betamethasone dipropionate, betamethasone valerate, clobetasol propionate, clobetasone butyrate, fluprednidene acetate, mometasone furoate). In some instnaces, the steroid is a labile prodrug ester (e.g. ciclesonide, cortisone acetate, hydrocortisone aceponate, hydrocortisone acetate, hydrocortisone buteprate, hydrocortisone butyrate, hydrocortisone valerate, prednicarbate, tixocortol pivalate). In some instances, the biologically active substances is daunorubicin. In some instances, the biologically active substance is colchicine. In some instances, the biologically active substance is a matrix metalloproteinase (MMP) inhibitor. In some instances, the MMP is prinomastat. In some instances, the biologically active substances is N-acetylcysteine. In some instances, one or more biologically active substances is low molecular weight heparin. In some instances, the concentration of low molecular weight heparin in the final hydrogel is approximately (5 IU/mL). In some instances, one or more biologically active substances is a non-steroidal anti-inflammatory drug (NSAIDS). In some instances, the NSAID is a salicylate (e.g. aspirin, diflunisal, salicylic acid, salsalate). In some instances, the NSAID is a propionic acid derivative (e.g. ibuprofen, dexibuprofen, naproxen, fenoprofen, ketoprofen, dexketoprofen, flurbiprofen, oxaprozin, loxoprofen). In some instances, the NSAID is an acetic acid derivative (e.g. indomethacin, tolmetin, sulindac, etodolac, detorolac, diclofenac, aceclofenac, bromfenac, nabumetone) In some instances, the NSAID is an enolic acid derivative (e.g. proxicam, meloxicam, tenoxicam, droxicam, lornoxicam, isoxicam, phenylbutazone). In some instances, the NSAID is an anthranilic acid derivative (e.g. mefenamic acid, meclofenamic acid, flufenamic acid, tolfenamic acid). In some instances, the NSAID is a selective cox-2 inhibitor (e.g. celecoxib, rofecoxib, valdecoxib, parecoxib, lumiracoxib, etoricoxib, firocoxib). In some instances, the NSAID is some other NSAID (e.g. nimesulide, clonixin, licofelone).

In other instances, one or more substances may result in an ultraconcentrated hydrogel with improved biologic activity, improved resident time, or reduced degradation, or reduced clearance. In some instances, one or more substances is collagen. In some instances, the collagen is fibrillar type (type I, II, III, V, or XI). In some instances, the collagen is non-fibrillar type (type IV, VI, VII, VIII, IX, X, XII, XIII, XIV, XV, XVI, XVII, XVIII, XIX, XX, or XXI). In some instances, the collagen is type I, type II, type III, type IV, or type V. In some instances, the substance is chondroitin sulfate. In some instances, the substance is a basement membrane component such as laminin or fibronectin. In some instances, the ultraconcentrated hydrogel contains a mixture of one or more of the aforementioned substances.

In some instances, the concentration of the one or more biologically active substances in the ultraconcentrated hydrogel is at or above 1 ug/mL, at or above 5 ug/mL, at or above 10 ug/mL, at or above 50 ug/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more biologically active substances are living cells. In some instances, these living cells are stem cells. In some instances, these cells are no longer alive but their cellular products (for example, but not limited to, growth factors).

In some instances, one or more biologically active substances increase the in vivo resident time of the ultraconcentrated hydrogel. Such substances include crosslinking agents that prevent degradation or slow the clearance of the hydrogel. In some instances, the pharmaceutical product is a crosslinking agent. Such cross-linking agents include 1,4-butanediol diglycidyl ether (BDDE), divinyl sulfone (DVS), 1,2,7,8-diepoxyoctane (DEO), hexamethylenediamine (HMDA), 1-ethyl-3-[3-diethylamino)propyl]carbodiimide (EDC).

In some instances, one or more biologically active substances may reduce the resident time of the hydrogel. In some instances, this reduction in hydrogel resident time is achieved by breakdown of the hydrogel by one or more biologically active substances. For instance, one or more biologically active substance may be hyaluronidase. In these instances, the hydrophilic polymer is hyaluronic acid or a derivative thereof. Hyaluronidase is added to the hydrophilic polymer powder or the aqueous excipient. In some variations, a hyaluronidase inhibitor is also added to prevent unexpected breakdown during storage. In some instances, this inhibitor is inactivated prior to use. In some instances, the effect of this inhibitor is reduced upon in vivo exposure.

In some instances, one or more biologically active substances may reduce intraocular pressure. In some instances, the biologically active substance that reduces intraocular pressure is a prostaglandin analog (e.g. latanoprost, bimatoprost, travoprost). In some instances, the biologically active substance that reduces intraocular pressure is a beta blocker (e.g. timolol, betaxolol). In some instances, the biologically active substance that reduces intraocular pressure is a parasympathomimetic (e.g. pilocarpine). In some instances, the biologically active substance that reduces intraocular pressure is a carbonic anhydrase inhibitor (e.g. dorzolamide, brinzolamide, acetazolamide). In some instances, the biologically active substance that reduces intraocular pressure is an adrenergic agent (e.g. brimonidine, apraclonidine).

In some instances, the ultraconcentrated hydrogel contains one or more substances to prevent degradation. In some instances, the ultraconcentrated hydrogel contains one or more substances to improve stability. In some instances, the ultraconcentrated hydrogel contains one or more substances to prevent infection. In some instances, the ultraconcentrated hydrogel contains one or more substances to reduce free radicals. In some instances, one or more of these substances is disodium-ethylene diamine tetra-acetate (EDTA), benzalkonium chloride (BAK), benzethonium chloride, sodium perborate (SP), chlorobutanol (Cbl), and/or stabilized thimerosal (Thi), phenylmercuric nitrate, vitamin C, vitamin E, butylatedhydroxyanisole (BHA), butylatedhydroxytoulene (BHT), propyl gallate, phenols, meta cresol, chloro cresol, parabens, methyl paraben, ethyl paraben, propyl paraben, butyl paraben, aryl acids, alkyl acids, benzyl alcohol, chlorobutanol, benzoic acid, sorbic acid, citric acid, bronopol, and/or propylene glycol.

In a first aspect, a syringe capable of storing an ultraconcentrated hydrogel. In some variations, the syringe is composed of a material designed for extended storage of a pharmaceutical product. In some variations, the syringe is designed to specifically storage one or more biologically active substances. In some variations, the container is composed of a polymer or copolymer. In some variations, the syringe is composed of cyclic olefin copolymer (COC). In some variations, the container is composed of cyclic olefin polymers (COP). In some variations, the cyclic olefin copolymer (COC) is composed of norbomnene. In some variations, the cyclic olefin copolymer (COC) is composed of tetracyclododecene. In some variations, the container is composed of polycarbonate. In some variations, the container is composed of polypropylene. In some variations, the container is composed of homo-polymer and/or co-polymer polypropylene. In some variations, the container is composed of metal. In some variations, the container is composed of stainless steel. In some variations, the container is composed of glass. In some variations, the container is composed of borosilicate glass. In some variations, the container is composed of a combination of one or more of the aforementioned materials. In some variations, the container is a syringe. In some variations, the container is considered a prefilled syringe. In some variations, the pre-filled syringe is designed for extended storage of a pharmaceutical product. In some variations, the syringe meets ISO 11040 guidelines.

In some variations, one or more components of the syringe are composed of a material that reduces oxygen permeation. In some variations, one or more components of the syringe are coated with a material that reduces oxygen permeation. In some variations, one or more components of the syringe are composed of a material that reduces water permeability. In some variations, one or more components of the syringe are coated with a material that reduces water permeability. In some variations, water vapor permeability is less than or equal to 0.1 (g×mm²)/(m²×d). In some variations, water absorption is less than 0.01%.

In some variations, the syringe plunger is composed of a thumb press, plunger rod, and plunger tip. In some variations, the plunger tip is composed of the same material as the plunger rod. In some variations, the plunger tip is composed of a different material than the plunger rod. In some variations, the plunger tip is designed to be compatible with the syringe coating. In some variations, the plunger tip is a cap-type design. In some variations, the plunger tip is an O-ring. In some variations, the plunger tip has one or more flanges. In some variations, the plunger tip screws into the syringe plunger rod. In some variations, the plunger tip is designed to reduce oxygen and/or water permeation. In some variations, the plunger tip is composed of rubber. In some variations, the rubber is a type of rubber validated for use in prefilled syringes. In some variations, the rubber is butyl rubber. In some variations, the rubber is bromobutyl rubber. In some variations, the rubber is a halobutyl rubber. In some variations, the rubber is ethylene propylene diene monomer rubber. In some variations, the plunger tip is composed of a polymer. In some variations, the plunger tip is composed of a polymer of isoprene. In some variations the plunger tip is composed of polyisoprene. In some variations, the plunger tip is composed of an organosilicon compound. In some variations, the organosilicon compound contains one or more functional siloxane groups. In some variations, the plunger tip is composed of silicone.

In some variations, the prefilled syringe has a cap closure. In some variations, the cap closure contains cyclic olefin copolymer (COC). In some variations, the syringe cap closure contains cyclic olefin polymers (COP). In some variations, the cyclic olefin copolymer (COC) is composed of norbornene. In some variations, the cyclic olefin copolymer (COC) contains tetracyclododecene. In some variations, the syringe cap closure contains rubber. In some variations, the rubber is butyl or bromobutyl rubber. In some variations, the rubber is halobutyl rubber. In some variations, the rubber is ethylene propylene diene monomer rubber. In some variations, the rubber is a combination of one or more types of rubber. In some variations, the syringe cap closure contains a polymer of isoprene. In some variations, the syringe cap closure contains polyisoprene. In some variations, the syringe cap closure composed of a material containing siloxane. In some variations, the O-ring is composed of silicone. In some variations, the cap is a combination of one or more of the above materials. In some variations, the syringe cap has a very low level of extractables.

In some variations, the syringe is designed to contain less than 1 mL of the ultraconcentrated hydrogel. In some variations, the syringe is designed to contain less than 0.5 mL of the ultraconcentrated hydrogel. In some variations, the syringe is designed to contain less than 0.3 mL of the ultraconcentrated hydrogel.

In some instances, the ultraconcentrated hydrogel has a concentration of greater than 100 mg/mL. In some instances, the ultraconcentrated hydrogel has a storage modulus G′ exceeding 160 Pa, preferably at least 180 Pa, more preferably at least 200 Pa. In some variations, the ultraconcentrated hydrogel contains other biologically active components such as antibodies, proteins, peptides, small molecules, aptamers, and/or cells (living or dead). In some variations, the ultraconcentrated hydrogel is hyaluronic acid.

In some variations, the syringe is sterilized prior to filling. In some variations, the syringe is sterilized after filling. In some variations, the syringe is aseptically filled. In some variations, the syringe is aseptically filled in a clean room. When sterilized after filling the hydrogel may undergo degradation during sterilization.

In another aspect, the syringe is designed to reduce the force required to inject (i.e. the injection force) the ultraconcentrated hydrogel through a microneedle to below a safe injection force threshold as defined by the Food and Drug Administration. Standard syringes containing ultraconcentrated hydrogels frequently require injection forces above 40 N. Such force poses significant safety issues when injecting into biologic tissue. In some variations, the reduction in injection force is below 10 N, below 15 N, below 20 N, below 25 N, or below 30 N. The syringe designed herein reduces the required injection force as compared to a standard syringe by at least 25%, by preferably at least 50%, or by most preferably at least 75%. Here a standard syringe is a syringe whose plunger tip has a diameter of 4 mm or greater and/or whose thumb press has a cross-sectional area that is less than 8-fold greater than a cross-sectional area of said plunger tip.

In some variations, injection force is reduced by reducing the internal syringe shaft diameter (and/or plunger tip). The relationship between the internal diameter of the syringe barrel and the diameter of the plunger tip is understood to match. In some variations, the syringe shaft diameter is less than 8 mm, less than 5 mm, preferably less than 4 mm, more preferably less than 3.5 mm, more preferably less than 3 mm, and more preferably less than 2 mm, and more preferably less than 1 mm. In some variations, injection force is reduced by increasing the surface area of the plunger thumb press. In some variations, the thumb press has a surface area of greater than 100 mm², greater than 150 mm², greater than 200 mm², greater than 250 mm², greater than 300 mm², greater than 350 mm², greater than 400 mm², greater than 500 mm². In some variations, injection force is reduced by both reducing the internal cross-sectional area of the syringe and increasing the surface area of the plunger thumb press. In such variations, the ratio of the surface area of the thumb press to the surface area of the plunger tip (or internal syringe cross sectional area) is greater than 10:1, preferably greater than 15:1, more preferably greater than 20:1, more preferably greater than 25:1, more preferably greater than 30:1, more preferably greater than 35:1, more preferably greater than 40:1, more preferably greater than 45:1, more preferably greater than 50:1.

In some variations, the syringe barrel is coated to reduce injection force. In some variations, the coating is applied in such a way so as to prevent leaching from the surface into the hydrogel. In some variations, the coating is applied such that the coating is covalently linked to the syringe barrel. In some variations, the covalently linkage is achieved through an ionizing gas plasma as is described in U.S. Pat. Nos. 8,084,103B2, 9,133,412B2, 7,431,989B2. In some variations, the coating is applied using ultraviolet. In some variations, the coating is applied using heat. In some variations, the coating is ‘baked’ on. In some variations, the syringe barrel is coated with a poly-siloxane-based compound In some variations, the syringe barrel is coated with dimethylpolysiloxane. In some variations, this poly-siloxane-based compound. In some variations, this coating is as described in U.S. Pat. No. 7,553,529B2. In some variations, the syringe barrel is coated with silicone oil In some variations, the coating contains both high and low molecular weight silicone oil. In some variations, the syringe barrel is coated with a fluorochemical compound. In some instances, the fluorochemical compound is a fluoropolymer. In some instances, fluorochemical compound is a perfluoropolyether. In some instances, the fluorochemical compound is a functionalized perfluoropolyether. In some instances, the coating is a combination of different compounds. In some instances, the lubricant contains additives such as free radical imitators, viscosity modifiers, surfactants, wetting agents, anticorrosive agents, antioxidants, antiwear agents, and/or buffering agents.

In some variations, the syringe has barrel flanges. In some variations, the syringe has finger grips. In some variations the barrel flanges are made of a different composite material as compared to the syringe body. In some variations, the barrel flanges are detachable. In some variations, the barrel flanges are made from a unibody construction that includes the syringe body. In some variations, the barrel flanges are made of a polymer. In some variations, the barrel flanges are made of cyclic olefin copolymer (COC). In some variations, the syringe cap is composed of cyclic olefin polymers (COP). In some variations, the cyclic olefin copolymer (COC) is composed of norbornene. In some variations, the cyclic olefin copolymer (COC) is composed of tetracyclododecene. In some variations, the barrel flanges are made of polycarbonate. In some variations, the barrel flanges are made of a polypropylene. In some variations, the barrel flanges are made of metal. In some variations, the barrel flanges are made of stainless steel.

In some variations, the syringe contains a method to prevent a needle from separating from the syringe during the injection. In some variations, the syringe contains a Leur lock. In some variations, an attachment is added after the needle is attached to the syringe to reduce the needle from dislodging from the syringe during injection. In some variations, this additional attachment may be separate from syringe itself. In some variations, the attachment to prevent needle dislodgement is made from plastic. In some variations, the attachment to prevent needle dislodgement is made from a polymer. In some variations, the attachment to prevent needle dislodgement is made from cyclic olefin copolymer (COC). In some variations, the attachment to prevent needle dislodgement is made from cyclic olefin polymers (COP). In some variations, the cyclic olefin copolymer (COC) is composed of norbornene. In some variations, the cyclic olefin copolymer (COC) is composed of tetracyclododecene. In some variations, the attachment to prevent needle dislodgement is made from polycarbonate. In some variations, the attachment to prevent needle dislodgement is made from of polypropylene. In some variations, the attachment to prevent needle dislodgement is made from poly(methyl methacrylate) (PMMA). In some variations, the attachment to prevent needle dislodgement is made from metal. In some variations, the attachment to prevent needle dislodgement is made from stainless steel. In some variations, the attachment to prevent needle dislodgement is made glass. In some variations, the attachment to prevent needle dislodgement is made from borosilicate glass. In some variations, both a leur lock and a separate attachment are added to prevent the needle from dislodging from the syringe during injection. In some variations, the attachment to prevent needle dislodgement is designed to encase the needle and the needle hub. In some variations, the needle is permanently attached to the syringe.

In some variations, the syringe can be attached to a small gauge needle for precision delivery into a biologic tissue. In some variations, this needle is specially designed to reduce the resistance of material passage, thereby further reducing the injection force. In some variations, the microneedle is designed to minimize force by increasing the internal diameter of the microneedle. In some variations, the internal diameter of the microneedle is greater than 300 um, greater than 400 um, or greater than 500 um. When injecting into biologic tissue, the outer diameter of the needle is limited to ideally less than 750 um for ocular indications and less than 1200 um for orthopedic indications. To maximize the internal diameter of the microneedle without expanding the outer diameter of the microneedle, the microneedle is designed to have an ultrathin wall thickness of less than 150 um, less than 125 um, less than 100 um, or less than 80 um. In some variations, the needle is coated to reduce friction. In some variations, the syringe barrel is coated with a poly-siloxane-based compound. In some variations, the syringe barrel is coated with dimethylpolysiloxane. In some variations, this poly-siloxane-based compound. In some variations, the needle is coated with silicone oil. In some variations, the coating contains both high and low molecular weight silicone oil. In some variations, the needle is coated with a fluorochemical compound. In some instances, the fluorochemical compound is a fluoropolymer. In some instances, fluorochemical compound is a perfluoropolyether. In some instances, the fluorochemical compound is a functionalized perfluoropolyether. In some instances, the needle is a combination of different compounds In some instances, the lubricant contains additives such as free radical imitators, viscosity modifiers, surfactants, wetting agents, anticorrosive agents, antioxidants, antiwear agents, and/or buffering agents. In some variations, the needle is coated with a fluoropolymer.

In some variations, the total syringe volume is 10 mL or less, is 7.5 mL or less, is 5 mL or less, is 2.5 mL or less, is 2 mL or less, is 1.5 mL or less, is 1 mL or less, is 0.75 mL or less, is 0.50 mL or less, is 0.25 mL or less, is 0.20 mL or less, is 0.1 mL or less.

In other variations, a method is disclosed that involves using the syringe containing the ultraconcentrated hydrogel to inject biologic tissue to treat diseases. In some variations, the ultraconcentrated hydrogel is hyaluronic acid. In such variations, the hyaluronic acid is ultraconcentrated at or above 5%, at or above 7.5%, at or above 10%, at or above 12.5%, at or above 15%, at or above 17.5%, at or above 20%, at or above 22.5%, at or above 25%, at or above 27.5%, at or above 30%, at or above 35%, at or above 40%, at or above 45%, at or above 50%, at or above 55%, at or above 60%. In some variations, the biologic tissue is the eye. In some variations, the biologic tissue is the vitreous cavity. In some variations, the injection of the ultraconcentrated hydrogel is used to treat ocular diseases. In some variations, these ocular diseases include, but are not limited to, exudative macular degeneration, dry macular degeneration, diabetic retinopathy, proliferative diabetic retinopathy, diabetic macular edema, retinal vein occlusion, cystoid macular edema, myopic degeneration, choroidal neovascular membrane, rhegmatogenous retinal detachment, tractional retinal detachment, exudative retinal detachment, infectious uveitis, inflammatory uveitis, Vogt-Koyanagi-Harada disease, white dot syndrome, glaucoma, primary open angle glaucoma, closed angle glaucoma, chronic glaucoma, ocular hypertension, myopia, intraretinal fluid, subretinal fluid, ruptured globe, hypotony, retinopathy or prematurity, coat's disease, and/or choroidal detachment. In some variations, these diseases are orthopedic diseases. In some variations, these diseases include disease involving joints. In some variations, these diseases involve inflammatory joint disease. Examples of inflammatory joint disease include, but are not limited to, rheumatoid arthritis, psoriatic arthritis, and ankylosing spondylitis. In some variations, these diseases involve age-related joint degeneration. In some variations, these diseases include osteoarthritis. In some variations, these diseases are dermatologic diseases. In some variations, these diseases related to aging. In some variations, these diseases are skin changes. In a preferred embodiment the disease is an ocular disease.

In some variations, the syringe is used to inject the ultraconcentrated hydrogel into the eye. In some variations, the syringe is used to inject the ultraconcentrated hydrogel into the vitreous cavity of the eye. In some variations, the syringe is used to inject the ultraconcentrated hydrogel into the anterior chamber of the eye. In some variations, the syringe is used to inject the ultraconcentrated hydrogel into the suprachoroidal space of the eye. In some variations, the syringe is used to inject the ultraconcentrated hydrogel into the subconjunctival and/or subtenon's space of the eye. In some variations, the syringe is used to inject the ultraconcentrated hydrogel into the joint space. In some variations, the syringe is used to inject the ultraconcentrated hydrogel into the dermal space. In some variations, the syringe is used to inject the ultraconcentrated hydrogel into a biologic implant located within a biologic tissue. In some variations, the implant is located or around the eye.

In some variations, the volume injected from the syringe into the biologic tissue is at or above 25 uL, is at or above 50 uL, is at or above 100 uL, is at or above 150 uL, is at or above 200 uL, is at or above 300 uL, is at or above 500 uL, is at or above 1 mL, is at or above 1.5 mL, is at or above 2.0 mL, is at or above 5 mL, is at or above 10 mL.

In some variations, the ultraconcentrated hydrogel injected from the syringe into the biologic tissue is hyaluronic acid with a concentration at or above 5% w/w, at or above 7.5% w/w, at or above 10%, at or above 12.5%, at or above 15%, at or above 17.5%, at or above 20%, at or above 22.5%, at or above 25%, at or above 27.5%, at or above 30%, at or above 32.5%, at or above 35%, at or above 37.5%, at or above 40%, at or above 45%, at or above 50%, at or above 55%, at or above 60%.

A method of preparing a hydrogel is described in U.S. provisional application No. 63/169,534 (Attorney docket No 003027USPROV) entitled METHODS FOR ASEPTIC MANUFACTURING OF HIGHLY CONCENTRATED HYDROGELS, the entire contents of which are hereby incorporated by reference.

In some variations, the ultraconcentrated hydrogel is injected from the syringe into the biologic tissue prior to injection with a biologically active substance. In some variations, the ultraconcentrated hydrogel is injected from the syringe into the biologic tissue after injection with a biologically active substance. In some variations, the ultraconcentrated hydrogel is injected from the syringe into the biologic tissue at the same time as injection with a biologically active substance. In some instances,

In an exemplary embodiment, a prefilled syringe containing a sterile ophthalmic pharmaceutical composition comprising hyaluronic acid with a molecular weight of 6.1 MDa in a carrier consisting of a balanced salt solution containing sodium chloride (6.4 mg/mL), potassium chloride (0.75 mg/mL), calcium chloride dihydrate (0.48 mg/mL), magnesium chloride hexahydrate (0.3 mg/mL), sodium acetate trihydrate (3.9 mg/mL), sodium citrate dihydrate (1.7 mg/mL), sodium hydroxide and/or hydrochloric acid (to adjust pH). In some exemplary embodiments, the carrier also contains the biologically acidic substance aflibercept, or a biosimilar thereof, at a concentration of 40 mg/mL. The ratio of hyaluronic acid to carrier is such that the sterile hyaluronic acid is present at 20% w/v. The pH of the solution is approximately 7.4 with an osmolality of approximately 320 mOsm/kg. Both the hyaluronic acid and carrier are sterile and aseptically mixed and filled into a sterile prefilled syringe The syringe body is composed of a syringe body composed of cyclic olefin polymer (COP). The barrel of the syringe has an internal diameter of 3.23 mm. The plunger tip is composed of ethylene propylene diene monomer (EPDM) rubber. The plunger rod has a thumb press with an approximate cross-sectional area that is 48 times larger than the surface area of the plunger tip. The prefilled syringe is coated with a silicone-based polymer that is covalently attached to the syringe barrel using a plasma. The prefilled syringe is then aseptically filled approximately 250 uL of the 20% hyaluronic acid described above and sealed in accordance with ISO 11040 guidelines. A 23-gauge needle is fabricated to be an ultra-thin walled needle with an outer wall diameter of approximately 0.654 mm and an inner wall diameter of approximately 0.5271 mm. The syringe shaft diameter, the surface area of the thumb press and plunger tip, the covalent coating on the syringe barrel, and the ultrathin 23-gauge needle reduce the injection force to below 20 newtons during injection into biologic tissue. In some exemplary embodiments, the prefilled syringe is used to inject 20% hyaluronic acid through the ultra-thin walled 23-gauge needle into the vitreous cavity of the eye to treat ocular conditions.

What is claimed as new and desired to be secured by Letters Patent of the United States is:

1. A pre-filled syringe comprising:
a syringe barrel;
a plunger comprising a plunger tip, a plunger rod, and a thumb press; and
a sterile pharmaceutical composition comprising a hydrogel at a concentration of at least 10% w/w (or w/v) and a pharmaceutically acceptable carrier having a storage modulus G′ of at least 160 Pa,
wherein an internal diameter of a syringe barrel is less than 4 mm; and
a cross-sectional area of said thumb press is at least 8-fold greater than an internal cross-sectional area of said syringe barrel.

2. The pre-filled syringe according to claim 1, wherein a diameter of the plunger tip is less than 4 mm.

3. The pre-filled syringe according to claim 1, wherein a cross-sectional area of said thumb press is at least 8-fold greater than a cross-sectional area of the plunger tip.

4. The pre-filled syringe according to claim 1, wherein said syringe barrel is comprised of a material selected from borosilicate glass or a cyclic-olefin copolymer.

5. The pre-filled syringe according to claim 1, wherein an inside surface of said syringe barrel comprise a covalently attached lubricant.

6. The pre-filled syringe according to claim 1 wherein said hydrogel is hyaluronic acid.

7. The pre-filled syringe according to claim 1, where a pressure necessary to eject said hydrogel through a needle is below 20N.

8. The pre-filled syringe according to claim 1, where a pressure necessary to eject said hydrogel through a needle is below 15N.

9. The pre-filled syringe according to claim 1, where a pressure necessary to eject said hydrogel through a needle is below 12N.

10. The pre-filled syringe according to claim 1, further comprising an ultrathin-walled microneedle attached thereto.

11. The prefilled syringe according to claim 8, wherein an ultrathin-walled microneedle has an inner diameter greater than 250 um.

12. The prefilled syringe according to claim 10, wherein the ultra-thin-walled microneedle bas an outer diameter of less than 670 um.

13. The prefilled syringe according to claim 1, wherein said hydrogel solution has a storage modulus G′ of at least 200 Pa.

14. The pre-filled syringe according to claim 1, wherein said syringe barrel further comprise an attachment to prevent a needle from separating.

15. The prefilled syringe according to claim 1, wherein said syringe barrel further comprises finger flanges.

16. The prefilled syringe according to claim 14, wherein said finger flanges resist a force greater than needed to eject said hydrogel.

17. The prefilled syringe according to claim 1, wherein the cross-sectional area of said thumb press is at least 10-fold greater than a cross-sectional area of said plunger tip.

18. The prefilled syringe according to claim 1, wherein the cross-sectional area of said thumb press is at least 15-fold greater than a cross-sectional area of said plunger tip.

19. The prefilled syringe according to claim 1, wherein the hydrogel solution contains a biologically active substance.

20. The prefilled syringe according to claim 1, wherein the hydrogel solution is crosslinked.
